(19) United States
(12) Patent Application Publication
(10) Pub. No.: US 2012/0178980 A1
(43) Pub. Date: Jul. 12, 2012

(54) Method of Making and Using a Hydrocarbon Conversion Catalyst

(75) Inventor: Jeffrey Amelse, Batavia, IL (US)

(73) Assignee: BP Corporation North America Inc., Naperville, IL (US)

(21) Appl. No.: 13/348,210

(22) Filed: Jan. 11, 2012

Related U.S. Application Data
(60) Provisional application No. 61/432,018, filed on Jan. 12, 2011.

(51) Int. Cl. C07C 15/08; B01J 29/00

(52) U.S. Cl. 585/435; 502/202

(57) ABSTRACT

Methods for making and using a catalytic composition useful in the hydrocarbon conversion reactions. The catalytic composition is made from an alumina sol that is prepared by dispersing a hydrated alumina in an aqueous medium. The alumina sol is mixed with a boron-containing molecular sieve. Catalytic compositions prepared in this manner avoid the disadvantages of preparing alumina sols via the Heard process.

This application claims the benefit of U.S. Provisional Patent Application No. 61/432,018 filed Jan. 12, 2011, which is incorporated herein by reference in its entirety.

### FIELD OF THE INVENTION

The present invention relates to a method of making and using a hydrocarbon conversion catalyst, and in particular, a method of making and using a catalyst composition made from a hydrated alumina and a boron-containing molecular sieve.

### BACKGROUND

Alumina containing catalysts have long been used in the chemical and refining industries for a variety of hydrocarbon conversion applications. Examples of such applications include the reforming of naphthas, the isomerization of alkylaromatics, and the transalkylation of alkylaromatics.

In one particular application, alumina containing catalysts have been used quite successfully in the production of paraxylene. Paraxylene is an important precursor in the production of polyester films and fibers. Paraxylene is made from a refinery feedstock containing predominately C₈ aromatic hydrocarbons. This C₈ aromatic hydrocarbon feedstock is sometimes referred to as a “mixed xylene feedstock,” and typically includes primarily orthoxylene, metaxylene, paraxylene, and ethylbenzene. Alumina containing catalysts have been used to isomerize the orthoxylene and metaxylene in mixed xylene feedstocks to form paraxylene. Some of these alumina catalysts have been developed to also simultaneously convert ethylbenzene to other aromatics which may be more readily separated from paraxylene product.

The alumina containing catalysts described above often contain silicon-containing compounds or boron-containing compounds. In one prior catalyst composition, the alumina serves as a binder in catalytic compositions having borosilicate molecular sieves. The borosilicate molecular sieves in such catalytic compositions may have low intrinsic activity for ethylbenzene conversion and xylene isomerization reactions. However, the sieves may become active upon placing the sieves in the alumina binder and removing water through evaporation and calcination.

One particular alumina and boron-containing catalyst composition is disclosed in U.S. Pat. No. 4,327,236. An example in this patent teaches the preparation of crystalline borosilicate molecular sieve on alumina catalysts by slurrying a crystalline borosilicate molecular sieve in an alumina sol which is designated as PHF alumina sol. Ammonium hydroxide is added to the slurry to form a gel, followed by drying and calcining. The use of the PHF alumina sol in the catalyst composition results in excellent conversions of mixed xylenes to paraxylene.

According to U.S. Pat. No. 4,664,781. PHF alumina sol, like many of the alumina sols in prior art catalyst compositions, is made by amalgamating high purity aluminum and then reacting the amalgamated metal with acetic acid containing water to produce an alumina sol. This is what is known as the “Heard process”. The Heard process and various improvements are described in U.S. Pat. No. 2,449,847, U.S. Pat. No. 2,686,159, U.S. Pat. No. 2,696,474, and U.S. RE 22,196.

The Heard process suffers some obvious disadvantages. The Heard process requires handling of mercury. The reaction of the metal and acetic acid forms liberated hydrogen gas and therefore must be performed in the substantial absence of oxygen. Both unreacted aluminum and mercury must be recovered from the reaction medium.

The use of a Heard-type alumina in making a borosilicate catalytic composition also has the disadvantage that the Heard-type alumina must be prepared with specialized equipment, often at a location distant from the where the catalytic composition is made. Since alumina made by the Heard process is in the form of a sol containing about 90% water, the cost of transportation and storage is significantly higher than it would be if the alumina was a dry powder. Furthermore, the Heard-type alumina sol can become unstable at low temperatures making it difficult to transport during winter months.

Despite all these disadvantages, it has long been thought that only Heard-type aluminas would sufficiently activate boron-containing molecular sieves to the extent necessary to achieve commercially relevant yields in certain chemical reactions, such as the isomerization of paraxylene. Accordingly, there remains a need to find alternative aluminas and alumina sols for use in boron-containing hydrocarbon catalytic compositions to achieve improved catalytic activity and product yields.

### SUMMARY

In one aspect of the invention, a method is provided for preparing a catalytic composition. An alumina sol is formed by dispersing a hydrated alumina in an aqueous medium. The sol is mixed with a boron-containing molecular sieve. Water is then removed from the sieve/sol mixture to form the catalytic composition.

According to another aspect of the invention, a method is provided for converting a hydrocarbon to at least one product. A feed stream containing a hydrocarbon is placed in the presence of a catalytic composition under reaction conditions suitable to chemically convert the hydrocarbon to at least one product. The catalytic composition is prepared by forming an alumina sol by dispersing a hydrated alumina in an aqueous medium. The sol is mixed with a boron-containing molecular sieve. Water is then removed from the sieve/sol mixture to form the catalytic composition.

According to another aspect of the invention, a method is provided for preparing a catalyst composition. A boron-containing molecular sieve is mixed with an alumina sol. The boron-containing molecular sieve is activated by heating the sieve/sol mixture. Water is then removed from the sieve/sol mixture.

The foregoing aspects are illustrative of those that can be achieved by the present invention and are not intended to be exhaustive or limiting of the possible advantages which can be realized. Thus, these and other aspects of the invention will be apparent from the description herein or can be learned from practicing the invention, both as embodied herein or as modified in view of any variation which may be apparent to those skilled in the art.

### DETAILED DESCRIPTION OF THE PREFERRED EMBODIMENTS

A method according to one embodiment of the present invention is directed to the preparation of a catalytic composition useful as a hydrocarbon conversion catalyst. The catalytic composition is prepared by forming an alumina sol by dispersing a hydrated alumina in an aqueous medium; mixing a boron-containing molecular sieve with the sol; and removing water from the sieve/sol mixture to form the catalytic composition.

As used herein, a “hydrated alumina” means either an aluminum oxide having bound thereto water of hydration, or a compound having an aluminum cation and one or more oxygen atoms and one or more hydrogen atoms. Examples of suitable hydrated aluminas include Al₂O₃.H₂0 (boehmite alumina), Al₇O₃.nH₂0, wherein 2>n>1 (pseudoboehmite alumina), alumina hydroxide in any of its forms such as gibbsite and bayerite, and aluminum oxide hydroxide in any of its forms such as diasopore, boehmite, and pseudoboehmite. In one particular embodiment, the hydrated alumina is either a boehmite or a pseudoboehmite alumina.

In one embodiment, the hydrated alumina used for making the sol is in a solid phase. The hydrated alumina may be in particle form, and in some embodiments, the hydrated alumina is in a powder form. It is advantageous to use hydrated alumina having small crystallite size and small aggregate particle size in order to provide greater surface area, which may in turn lead to increased activity. In one embodiment, the particles of the hydrated alumina preferably have an average surface area of at least 200 m²/g, and more preferably at least 230 m²/g, even more preferably at least 260 m²/g, and even more preferably at least 270 m²/g. In another embodiment, the particles of the hydrated alumina preferably have an average surface area of at least 280 m²/g, and more preferably at least 300 m²/g. Average surface area may be determined by any known method, such as by a BET method.

In one embodiment of the invention, the hydrated alumina has an alumina content of at least 50 wt %, and more preferably at least 60 wt %, and even more preferably at least 65 wt %. In another embodiment, the hydrated alumina is at least 70 wt % alumina.

It is advantageous to use relatively pure hydrated alumina in preparation of the catalyst composition to achieve catalyst compositions of high catalytic activity. The relatively pure hydrated aluminas used to make catalyst composition preferably have a low alkali metal content, because alkali metals may poison the active sites in the catalytic composition. In one embodiment, the hydrated aluminas have an alkali metal content of less than 100 ppm by weight, and more preferably less than 50 ppm by weight. In another embodiment, the hydrated alumina has an alkali metal content of less than 25 ppm by weight.

The hydrated alumina particles or powder may, however, include, or have amounts added thereto of, an acid to assist in dispersing the hydrated alumina in the aqueous medium. Suitable acids include monovalent mineral or organic acid, such as acetic acid, nitric acid, formic acid, tartaric acid, and citric acid, among others. These acids may be added to the hydrated alumina particles by impregnation or other methods known to those skilled in the art. Hydrated aluminas that are “preloaded” with such acids achieve high dispersion in water and exhibit less solids settling without the need to add additional acid. In one embodiment of the invention, the hydrated alumina includes at least 2 wt % acetic acid, and more preferably at least 3 wt % acetic acid, more preferably at least 4 wt % acetic acid, and even more preferably at least 5 wt % percent acetic acid. In another embodiment, the hydrated alumina includes from 2.0 wt % to about 8.0 wt %, and more preferably, 5.5 wt % to 7.5 wt % acetic acid. In another embodiment, the hydrated alumina includes at least 2 wt % nitric acid, and more preferably at least 3 wt % nitric acid, more preferably at least 4 wt % percent nitric acid. In another embodiment, the hydrated alumina includes from 2 wt % to 5 wt % nitric acid, and preferably 3 wt % to 4 wt % nitric acid. One suitable hydrated alumina preloaded with acetic acid is DISPERAL P3 alumina sold by Sasol North America of Houston, Tex. One suitable hydrated alumina preloaded with nitric acid is DISPERAL P2 alumina also sold by Sasol North America of Houston, Tex. DISPERAL is a registered trademark of Sasol Germany Gmbh of Hamburg, Germany.

A hydrated alumina suitable for use in the present invention may be made by any of a number of known methods in the art. For example, hydrated alumina may be made by the hydrolysis of aluminum alkoxides, as a byproduct in what is known as the Ziegler process. As described in the section on Aluminum Oxide (Alumina) Hydrated in the Kirk Othmer Encyclopedia of Chemical Technology, the Ziegler process involves the formation of aluminum alkoxides at intermediate stages. Hydrolysis of the alkoxides produces aluminum hydroxide having a pseudoboehmite structure. The hydroxide product is further processed to remove residual alcohols and then dried. The chemical purity of alumina powders produced this way is generally high, and in particular, alkali metal content of these aluminas is generally very low, although the aluminas sometimes may have some degree of titanium impurities. Hydrated aluminas may also be made by the reaction of aluminum and alcohols, which produces alumina of very high purity and very low traces of titanium.

The aqueous medium used for making alumina sol may be pure or substantially pure water. However, in one embodiment, acid is added to the aqueous medium to assist in the dispersion of the hydrated alumina. Suitable acids include monovalent mineral or organic acid, such as acetic acid, nitric acid, formic acid, tartaric acid, and citric acid, among others. In one embodiment, the acid concentration of the aqueous medium is at least 0.1 wt % and more preferably at least 0.3 wt %, and more preferably at least 0.6 wt %. The acid concentration in the aqueous is advantageously less than 3 wt %, and preferably less than 1.2 wt %. In one particular embodiment, the aqueous medium has an acetic acid concentration of 0.3 wt % to 1.2 wt %, more preferably 0.6 to 1.0 wt %. In another particular embodiment, the aqueous medium has a nitric acid concentration of 0.3 wt % to 1.2 wt %, more preferably 0.6 to 1.0 wt %

The alumina sol is prepared by adding the hydrated alumina to the aqueous medium while stirring. Notably, the alumina sol is prepared by a method other than the Heard process. In one embodiment, the alumina sol is prepared without reacting aluminum metal with acetic acid. In another embodiment, the alumina sol is prepared without the use of mercury. In another embodiment, the alumina sol is also prepared without the use of an amalgamated aluminum. In some embodiments, the alumina sol is prepared without the release of hydrogen gas.

Any of a number of boron-containing molecular sieves may be used in the catalytic compositions of the present invention. The resulting catalytic compositions include 5 wt % to 80 wt % borosilicate material, and 20 wt % to 95 wt % alumina. One particular suitable borosilicate is described in U.S. Pat. No. 4,327,236. Another example of a suitable borosilicate is the AMS-1B, described in U.S. Pat. No. 4,269,813. Another suitable borosilicate is the hydrogen form of AMS-1B, known as HAMS-1B.

A suitable AMS-1B crystalline borosilicate generally can be prepared by mixing an aqueous medium of oxides of boron, an alkali metal or an alkaline earth metal, such as sodium, and silicon, together with alkylammonium cations or a precursor of alkylammonium cations, such as an alkylamine, an alkylamine plus an alkyl hydroxide, an alkylamine plus an alkyl halide, or an alkylamine plus an alkyl acetate. The alkyl groups in the alkylammonium cations may be the same, or mixed, such as tetraethyl, or diethyl-dipropyl-ammonium cations. The mole ratios of the various reactants can be varied considerably to produce the AMS-1B crystalline borosilicates. The AMS-1B crystalline borosilicate can also be prepared in substantial absence of a metal or ammonium hydroxide as described in U.S. Pat. No. 5,053,211.

In another embodiment, the boron-containing molecular sieve is a borosilicate molecular sieve having the MFI framework type, as designated by the Structure Commission of the International Zeolite Association. In another embodiment the boron-containing molecular sieve also includes aluminum and thus may be known as a boroaluminosilicate molecular sieve.

The boron-containing molecular sieve is mixed with the alumina sol by stirring to form the sieve/sol mixture. Water may then be removed from the sieve/sol mixture in any of the methods known to those skilled in the art, such as calcining or evaporating. The sieve may be mixed with the alumina sol while stirring at ambient temperature or at an elevated temperature.

In one embodiment, water may be removed from the sieve/sol mixture by calcining. Calcining of the mixture is performed at 800° F. to 1100° F., for about 1 to 24 hours. In one embodiment, calcining is performed at 900° F. to 1000° F. for about 2 to about 6 hours. In other embodiments, water may also be evaporated prior to calcining. Evaporation occurs at elevated set point temperatures of 200° F. to 400° F. for about 1 to 24 hours. In one embodiment, the water in the sieve/sol mixture is removed by drying at a set point of from 200° F. to about 400° F., and more preferably between from about 325° F. to about 400° F. The vessel or tray holding the sieve/sol may be uncovered during evaporation, or may be at least partially covered.

The boron-containing molecular sieve is typically activated during the removal of water from the sieve/sol mixture by the evaporation and/or calcining. Activation as used herein means altering the sieve or its environment in some manner such that the catalyst compositions including the sieves have a higher catalytic activity than they did prior to such activation. However, according to another embodiment of the invention, the boron-containing molecular sieve may be activated by heating the sieve/sol mixture prior to water removal. The heat may be elevated for a time period before raising the temperature even higher to begin the evaporation and calcination. In this embodiment, the sieve/sol mixture is heated to a temperature of less than 100° C. to affect activation without significant evaporation of water. The sieve/sol mixture is heated to at least 50° C., and more preferably between 70 and 90° C. The temperature of the sieve/sol mixture may also be ramped down after activation and prior to water removal. Activation prior to water removal has the advantage of eliminating the variability of the activity that may be caused by particular drying and calcining procedures.

The sieve/sol mixture may also be gelled prior to calcination and/or evaporation. In one embodiment, the sieve/sol mixture is gelled by adding a gelling agent to the sieve/sol mixture prior to removal of water from the mixture. In another embodiment, the sieve/sol mixture is gelled after heating of the sieve/sol mixture to activate the boron-containing compound. One suitable gelling agent is concentrated ammonium hydroxide added in amounts of about 0.5 to about 1.5 cc (nominal 28-30 wt % ammonia) per gm of alumina solids present after drying and calcining the alumina sol. Other suitable gelling agents are known to those in the art. In one embodiment, suitable gelling agents include other coagulating salts such as ammonium chloride, ammonium nitrate, ammonium citrate, ammonium acetate, ammonium oxalate, ammonium tartrate, and ammonium carbonate.

Catalytically-active metals may also be added to the catalytic composition individually or in combination. Catalytically active metals may provide a hydrogenation-dehydrogenation function to the catalyst composition. Catalytically-active metals include, but are not limited to, tungsten, vanadium, molybdenum, rhenium, nickel, cobalt, chromium, or a noble metal, such as platinum or palladium. Such metals may be incorporated into the catalytic composition by impregnation and/or cation-exchange techniques known to those skilled in the art. In one embodiment, the metal is added to the sieve/sol mixture after gelling but before calcination. In another embodiment, the metal is added after drying and calcining the gelled sieve/sol mixture.

The catalytic compositions prepared according to the present invention may be used in any of a number of hydrocarbon conversion reactions. Examples include fluidized catalytic cracking; hydrocracking; the isomerization of normal paraffins and naphthenes; the reforming of naphthas and gasoline-boiling-range feedstocks; the isomerization of aromatics, especially the isomerization of alkylaromatics, such as xylenes; the disproportionation of aromatics, such as toluene, to form mixtures of other more valuable products including benzene, xylene, and other higher methyl substituted benzenes; hydrotreating; alkylation; and hydrodealkylation. The AMS-1B borosilicates, in certain ion-exchanged forms, can be used to convert alcohols, such as methanol or ethanol, to useful products, such as aromatics or olefins.

A process according to one embodiment of the invention includes a feed stream containing a hydrocarbon in the presence of a catalytic composition prepared according to the present invention and under reaction conditions suitable to chemically convert the hydrocarbon to at least one product. In one embodiment, the feed stream includes an alkylaromatic compound, and the product is an isomer of the alkylaromatic compound. In one particular embodiment, the feed stream includes C₈ aromatics, or mixed xylenes, including orthoxylene, metaxylene, paraxylene, and ethylbenzene. The mixed xylene feed stream is reacted at ethylbenzene conversion/xylene isomerization conditions to form a product stream containing a higher concentration of paraxylene than in the feed stream. Reaction may take place in the liquid, vapor or gaseous (supercritical) phase in the presence or substantial absence of hydrogen. Typical vapor phase reaction conditions comprise a temperature of from about 500° F. to about 1000° F., a pressure of from about 0 psig to about 500 psig, an H₂/hydrocarbon mole ratio of from about 0 to 10, and a liquid weight hourly space velocity (LWHSV) of from about 1 to about 100. Preferred vapor phase reaction conditions for xylene isomerization in a commercial paraxylene plant comprise a temperature of from about 600° F. to about 850° F., a pressure of from about 100 to about 300 psig, an H₂/hydrocarbon mole ratio of from about 0.5 to about 4, and a LWHSV of from about 5 to about 15. Typical and preferred vapor phase conditions for ethylbenzene conversion/xylene isomerization are further described, for example, in U.S. Pat. No. 4,327,236. Typical and preferred liquid phase conditions for ethylbenzene conversion/xylene isomerization are described, for example, in U.S. Pat. No. 4,962,258. Typical and preferred conditions for ethylbenzene conversion/xylene isomerization at supercritical temperature and pressure conditions are described, for example, in U.S. Pat. No. 5,030,788.

According to another embodiment of the invention, a catalytic composition is prepared by a method according to the present invention.

The novel methods of preparing an alumina containing catalytic composition according to the present invention avoids the disadvantages of using alumina sols prepared by the Heard process. Alumina sols made by the Heard process typically contain only around 10 wt % alumina solids, and therefore 90 gm of solution must be transported to the catalyst manufacturer per 100 gm of sol. In the method described here, only the weight of the hydrated alumina solids need be transported and stored. The method according to the present invention also avoids the necessity of handling the pre-amalgamated metal in the substantial absence of oxygen. The method of the present invention also avoids need to handle mercury, as well as eliminating the necessity of have to recover mercury and unreacted aluminum in the reaction mixture.

The methods of the present invention also unexpectedly provide hydrocarbon conversion yields similar to those provided by the Heard process, as demonstrated by the Example 1:

### Example 1

### Comparison of Catalyst Yields

The catalysts referred to below were tested for activity in isomerizing the xylene isomers in a pilot plant having a vapor phase fixed bed reactor. Approximately 4 gm of catalyst was used in each run. Approximately 2 gm of 2 wt % molybdenum on alumina was used as a guard bed on top of the catalyst. The mixed xylenes feed had a composition in Wt % of:

| | |
|---|---|
| Non-aromatics | 3.03 |
| Benzene | 0.46 |
| Toluene | 3.34 |
| Ethylbenzene | 6.00 |
| Paraxylene | 9.70 |
| Metaxylene | 52.21 |
| Orthoxylene | 23.53 |
| C₉ Aromatics | 1.57 |
| C10+ Aromatics | 0.17 |

The test conditions were approximately as follows:

| | | |
|---|---|---|
| Temperature | 600 | F. |
| Pressure | 250 | psig |

| | |
|---|---|
| H2/Hc mole ratio | 1.5 |
| LWHSV | 38 |

These conditions were chosen so that catalysts could be compared based on their activity for isomerizing the xylene isomers, and not conditions that are optimal for ethylbenzene conversion. However, in all cases, some conversion of ethylbenzene was observed. In most cases, the isomerization of the xylene isomers can be the more technically challenging reaction relative to ethylbenzene conversion.

At very high catalyst activity or very high reactor severity (such as high reactor temperature or low LWHSV) the xylene isomers will approach equilibrium. The reactor conditions for catalyst ranking were chosen such that the xylenes were far enough below equilibrium for a reference catalyst such that it is possible to determine if the new catalysts are of higher or lower activity for the isomerization of the xylene isomers. The measure of activity for this reaction is the weight percentage of paraxylene among all the xylenes (XYL), including paraxylene (pX), metayxlene (mX), and orthoxylene (oX) in the reactor effluent, calculated as % pX/XYL=(Wt % pX/(Wt % pX+Wt % mX+Wt % oX) At this temperature the equilibrium value of % pX/XYL is around 24%. A higher value of % pX/XYL means higher activity for the isomerization of the xylene isomers. The % pX/XYL in the reactor effluent is reported for the second day of operation at the reactor conditions stated above (within the ability to control the reactor at those conditions).

This example illustrates typical preparation conditions for a prior art catalyst comprising a HAMS-1B borosilicate molecular sieve on an alumina binder with the source of being PHF alumina available from Criterion Catalysts and Technologies of Houston, Tex. HAMS-1B refers to the hydrogen form of AMS-1B.

20 gm of HAMS-1B commercially prepared borosilicate molecular sieve was first slurried in 60 gm of deionized and distilled water. This slurry was homogonized. This homogenized slurry was added to 800 gm of PHF alumina sol having a solids content of 10.1 wt % and vigorously mixed for 5 minutes. This mixture was then gelled by adding 80 cc of concentrated ammonium hydroxide (28-30% ammonia). Mixing continued for 5 minutes. The gel was transferred to a glass dish and then dried for 4 hours at 328 F, ramped to 900 F over 4 hours, and then calcined at 900 F for 4 hours.

This catalyst has a nominal overall composition of approximately 20 wt % HAMS-1B and 80 wt % alumina binder. It is designated herein as Prior Art Catalyst X.

A commercially prepared catalyst comprising a nominal overall composition of approximately 20 wt % commercial HAMS-1B and 80 wt % PHF alumina binder was chosen as another reference. It is designated herein as Prior Art Catalyst Y.

These two catalysts were tested in a pilot plant for activity for isomerizing the xylene isomers with the mixed xylenes feed and at the reactor conditions stated above.

Prior Art Catalyst X achieved a % pX/XYL of 22.35%.

Prior Art Catalyst Y achieved an average % pX/XYL of 23.00% for 6 runs. For these six runs, the highest measured % pX/XYL was 23.24%. The lowest measured % pX/XYL was 22.77%. The range indicates precision of the measurement that can be expected due to slight deviations from reaction conditions and/or catalyst deactivation during the course of the testing.

For comparisons purposes, aluminas according to the present invention were also prepared.

100.3 gm of DISPERAL P3 alumina powder (available from Sasol North America, Houston Tex.) was dispersed in 900.4 gm of a 0.6 wt/0 acetic acid solution, while stirring for 15 minutes to form an alumina sol. The sol had a pH of 4.2. It was aged at room temperature for 2 hours. No settling of solids was observed indicating high dispersibility of the alumina powder.

20.0 gm of a commercially prepared HAMS-1B sieve was slurried in 60.0 gm of deionized and distilled water. This mixture was homogenized for 3 minutes and left to sit at room temperature for an additional one minute.

800.9 gm of the alumina sol (80 gm nominal DISPERAL P3 alumina solids) was added to the sieve in water mixture, and this mixture was homogenized for 5 minutes and left to sit for 30 minutes. This HAMS-1B sieve in alumina sol mixture was transferred to a mixer, and while stirring, was gelled with 80 ml of concentrated ammonium hydroxide (gelation ratio=1 cc concentrated ammonium hydroxide per gm alumina solids). This mixture was mixed for 5 minutes and then transferred to a glass dish. The mixture was dried for 4 hours at 329 F, ramped to 900 F over 4 hours, and then calcined at 900 F for 4 hours.

This catalyst was labeled Catalyst A. It has a nominal overall composition of 20 wt % sieve and 80 wt % alumina binder.

Other catalysts were prepared according to the general method of the present invention as described above with respect to Catalyst A, except the acid concentration of the aqueous medium and gelation ratio were altered. The Catalyst are designated herein as Catalysts B through H. These catalysts all have a nominal overall composition of about 20 wt % HAMS-1B-3 and 80 wt % alumina binder. All are prepared from DISPERAL P3 alumina powders prepared via aluminum alkoxides intermediates.

The results are summarized in the following table,

TABLE 1

| | Acid conc. | Gelation | % pX/% XYL in |
|---|---|---|---|
| Catalyst ID | (wt %) | Ratio | Reactor Effluent |
| Prior Art X | unknown | 1 | 22.35 |
| Prior Art Y | unknown | unknown | 23.00 |
| A (first run) | 0.6 | 1 | 22.92 |
| A (second run) | 0.6 | 1 | 22.93 |
| B | 0.6 | 0.75 | 23.18 |
| C (first run) | 0.6 | 2 | 22.98 |
| C (second run) | 0.6 | 2 | 22.95 |
| D | 0.3 | 0.75 | 22.68 |
| E | 1.2 | 0.75 | 22.21 |
| F | 0.3 | 1.5 | 21.97 |
| G | 1.2 | 1.5 | 21.40 |

Comparing the % pX/XYL for catalysts in this table to that achieved by the results achieved by catalysts prepared according to the prior art, several conclusions can be made: Catalyst prepared according to the methods of this invention can have activities comparable or higher than catalysts prepared according to the prior art using alumina sols prepared by the Heard process. Of the catalysts illustrated in this example, best results were obtained using DISPERAL P3 alumina dispersed in 0.6 wt % acetic acid and a gelation ratio of 0.75. However, good results were obtained with DISPERAL P3 alumina when dispersed in a range of acetic acid concentrations and even in water with no additional acid and when using a range of gelation ratios.

### Example 2

### Activation of Boron-Containing Molecular Sieve

A catalyst according to the present invention was prepared by adding 200.0 gm of DISPERAL P3 alumina to 1800 gm 0.6 wt % acetic acid. 40 gm of HAMS-1B-3 sieve was added to 120.0 gm D&D water. 1600 gm of this mixture was added to a 6 liter flask. The mixture was heated to 80 C for 1 hour. After one hour, the heating was stopped, and the mixture gelled by adding 120 ml of concentrated ammonium hydroxide. The gel first thickened, but then thinned. The gel was then split into three portions. 869.8 gm of the gel was dried at 329 F for 4 hours, ramped to 900 F over 4 hours, then calcined at 900 F for 4 hours. The Catalyst was screened for xylene isomerization activity at T=602.1 F, P=250 psig, H2/Hc=1.51, LWHSV=38.35 using the same feed used in Example 1. The reactor effluent had a pX/(pX+mX+oX) of 22.12% for cut 2 at 1.6 days-on-stream.

It should be readily understood by those persons skilled in the art that the present invention is susceptible of a broad utility and application. Many embodiments and adaptations of the present invention other than those herein described, as well as many variations, modifications and equivalent arrangements will be apparent from or reasonably suggested by the present invention and the foregoing description thereof, without departing from the substance or scope of the present invention.

Accordingly, while the present invention has been described herein in detail in relation to specific embodiments, it is to be understood that this disclosure is only illustrative and exemplary of the present invention and is made merely for purposes of providing a full and enabling disclosure of the invention. The foregoing disclosure is not intended or to be construed to limit the present invention or otherwise to exclude any such other embodiments, adaptations, variations, modifications and equivalent arrangements, the present invention being limited only by the claims appended hereto and the equivalents thereof.

1. A method of preparing a catalytic composition comprising:
forming an alumina sol by dispersing a hydrated alumina in an aqueous medium;
mixing a boron-containing molecular sieve with the sol; and
removing water from the sieve/sol mixture.

2. The method of claim 1, wherein the aqueous medium comprises at least about 0.3 wt % of an acid.

3. The method of claim 2, wherein the acid comprises acetic acid.

4. The method of claim 2, wherein the acid comprises nitric acid.

5. The method of claim 1, wherein the alumina sol is prepared without reacting aluminum metal with acetic acid.

6. The method of claim 1, wherein the alumina sol is prepared without the use of mercury.

7. The method of claim 1, wherein the alumina sol is prepared without the use of an amalgamated aluminum.

8. The method of claim 1, wherein the hydrated alumina comprises a boehmite alumina.

9. The method of claim 1, wherein the hydrated alumina comprises a pseudoboehmite alumina.

10. The method of claim 1, wherein the hydrated alumina comprises an aluminum hydroxide.

11. The method of claim 1, wherein the hydrated alumina comprises an aluminum oxide hydroxide.

12. The method of claim 1, wherein the boron-containing molecular sieve comprises a MFI framework.

13. The method of claim 1, wherein the boron-containing molecular sieve comprises a borosilicate compound.

14. The method of claim 13, wherein the borosilicate compound comprises AMS-1B or HAMS-1B.

15. The method of claim 1, wherein removing water from the sieve/sol mixture comprising calcining the sieve/sol mixture.

16. The method of claim 15, further comprising gelling the sieve/sol mixture prior to calcining.

17. The method of claim 15, further comprising evaporating water from the sieve/sol mixture at an elevated temperature prior to calcining.

18. The method of claim 17, wherein the sieve/sol mixture is at least pa covered while evaporating water from the sieve/sol mixture at an elevated temperature.

19. The method of claim 1, further comprising activating the boron-containing molecular sieve prior to removing water from the sieve/sol mixture.

20. The method of claim 19, wherein activating the boron-containing molecular sieve comprises heating the sieve/sol prior to removing water from the sieve/sol mixture.

21. The method of claim 20, wherein the temperature of the sieve/sol mixture is ramped down for a period after activating the boron-containing molecular sieve and prior to removing water from the sieve/sol mixture.

22. The method of claim 1, wherein said hydrated alumina is made by the hydrolysis of aluminum alkoxides.

23. The method of claim 1, wherein said hydrated alumina is made by the reaction of aluminum and alcohols.

24. The method of claim 1, wherein the hydrated alumina comprises at least 50 wt % alumina.

25. The method of claim 1, wherein the hydrated alumina comprises at least 65 wt % alumina.

26. The method of claim 1, wherein the hydrated alumina comprises at least 70 wt % alumina.

27. The method of claim 1, wherein the hydrated alumina includes at least 3 wt % acetic acid.

28. The method of claim 1, wherein the hydrated alumina includes at least 2 wt % nitric acid.

29. The method of claim 1, wherein the hydrated alumina includes less than about 50 ppm by weight alkali metals.

30. The method of claim 1, wherein the hydrated alumina comprises particles having an average surface area of at least 200 m²/g.

31. A method for converting a hydrocarbon, comprising reacting a feed stream containing a hydrocarbon in the presence of a catalytic composition prepared according to the method claim 1, and under reaction conditions suitable to chemically convert the hydrocarbon to at least one product.

32. The method of claim 31, wherein the hydrocarbon comprises an alkylaromatic compound.

33. The method of claim 32, wherein the at least one product comprises an isomer of the alkylaromatic compound.

34. The method of claim 33 wherein the alkylaromatic compound comprises paraxylene.

35. A method for producing paraxylene, comprising reacting a feed stream containing mixed xylenes in the presence of a catalytic composition prepared according to the method claim 1, and under reaction conditions suitable to form a product stream containing paraxylene in a concentration greater than the feed stream.

36. A method for preparing a catalyst comprising,
mixing a boron-containing molecular sieve with an alumina sol;
activating a boron-containing molecular sieve by heating the sieve/sol mixture; and
removing water from the sieve/sol mixture.

37. The method of claim 36, wherein the activation of the sieve/sol mixture occurs at a temperature of less than 100° C.

38. The method of claim 36, wherein the activation of the sieve/sol mixture occurs at a temperature of greater than 50° C.

39. The method of claim 36, wherein removing water from the sieve/sol mixture comprises calcining the sieve/sol mixture.

40. The method of claim 39, further comprising gelling the sieve/sol mixture prior to calcining.

41. The method of claim 39, further comprising evaporating water from the sieve/sol mixture at an elevated temperature prior to calcining.

42. The method of claim 41, wherein the sieve/sol mixture is at least partially covered while evaporating water from the sieve/sol mixture at an elevated temperature.

43. The method of claim 36, further comprising lowering the temperature for a time period after the activation of the sieve/sol mixture and before removal of water from the sieve/sol mixture.

44. A catalyst composition made from the method of claim 1.

45. A catalyst composition made from the method of claim 36.
